(19) United States
(12) Patent Application Publication
(10) Pub. No.: US 2003/0031643 A1
(43) Pub. Date: Feb. 13, 2003

(54) Composition in the form of a water-in-oil emulsion stabilized with a 2-acrylamido-2-methylpropanesulphonic acid (CO) polymer containing hydrophobic grafts

(75) Inventors: Florence L'alloret, Paris (FR); Odile Aubrun-Sonneville, Antony (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: L'OREAL, Paris (FR)

(21) Appl. No.: 10/166,128

(22) Filed: Jun. 11, 2002

(30) Foreign Application Priority Data
Jun. 11, 2001 (FR) 01 07609

(51) Int. Cl. A61K 7/06; A61K 7/11

(52) U.S. Cl. 424/70.16; 424/70.17

(57) ABSTRACT

A composition, in the form of a water-in-oil emulsion, is characterized in that it contains at least one amphiphilic polymer or copolymer of 2-acrylamido-2-methylpropanesulphonic acid bearing hydrophobic grafts chosen from hydrocarbon-based radicals containing from 6 to 30 carbon atoms, oxyalkylenated hydrocarbon-based radicals comprising a hydrocarbon-based group containing from 6 to 30 carbon atoms and at least one oxyalkylene unit, and mixtures thereof.

### BACKGROUND OF THE INVENTION

1. Field of the Invention

The present invention relates generally to a composition, preferably a cosmetic and/or dermatological composition in the form of a water-in-oil emulsion, preferably free of emulsifying surfactant or containing only a small amount of emulsifying surfactant, comprising an aqueous phase stabilized in an oily phase and a 2-acrylamido-2-methylpropanesulphonic acid amphiphilic polymer or copolymer preferably which is liposoluble or lipo-dispersible or soluble or dispersible in a polar solvent. Methods of preparing and using these compositions are also disclosed and make up a part of the invention.

2. Background of the Invention

Water-in-oil emulsions are dispersions of aqueous globules in a continuous oily phase. These emulsions are generally stabilized with amphiphilic molecules of low molar mass (<5000 g/mol) such as alkylglycerolated compounds, alkylpolyoxyethylenated compounds or silicone compounds. These emulsions have the following drawbacks:

the use of emulsifying surfactants that are relatively aggressive with respect to the skin, and present at concentrations of greater than 1% (by weight);

they are difficult to stabilize because the repulsive forces between drops, of steric origin, are of low range. This is reflected by a phenomenon of flocculation, which can give rise to sedimentation and/or even coalescence;

silicone amphiphilic compounds are specific to oils of the same nature, which limits the range of oils that may be used.

Silicone polymers for stabilizing water-in-oil emulsions also exist; in this case, the oily phase must contain a predominant amount of a silicone oil.

Patent application EP 1 069 142 A1 relates to water-soluble polymers obtained by free-radical polymerization of at least one macromonomer comprising a polyoxyalkylenated hydrophilic block and a hydrophobic block that can contain from 1 to 30 carbon atoms with at least one olefinically unsaturated comonomer comprising oxygen, nitrogen, sulphur, phosphorus, chlorine and/or fluorine, in particular acrylamidomethylpropanesulphonic acid. These polymers may be used as thickeners, emulsifiers, dispersants, suspension agents, stabilizers and/or consistency agents for an aqueous preparation, especially in the cosmetic and/or pharmaceutical fields.

### OBJECT OF THE INVENTION

There is thus a need for a stable composition in the form of a water-in-oil (W/O) emulsion, that can be used especially in cosmetics and/or dermatology and that:

contains small amounts of emulsifying surfactant (that is to say contains 1% or less by weight of emulsifying surfactant relative to the total weight of the composition), preferably less than 1% by weight, and better still contains none at all;

can contain hydrocarbon-based oils in large amounts (that is to say 5% or more by weight relative to the total weight of the composition); and

is stable with respect to flocculation.

### BRIEF DESCRIPTION OF THE DRAWINGS

FIGS. 1 and 2 show the water-in-oil emulsion obtained in Example 1 at various magnifications.

### DETAILED DESCRIPTION OF THE INVENTION

The inventors has discovered, surprisingly, that polymers and copolymers derived from 2-acrylamido-2-methylpropanesulphonic acid (AMPS) and bearing hydrophobic grafts chosen from C₆-C₃₀ hydrocarbon-based radicals, the C₆-C₃₀ hydrocarbon-based radicals comprising at least one oxyalkylene unit, prefluoroalkyl radicals, silicon radicals, and mixtures thereof, make it possible to obtain water-in-oil emulsions that do not flocculate and that can contain large amounts of hydrocarbon-based oils. The molar degree of grafting with hydrophobic graft of the polymers is greater than or equal to 50% (50% or more), preferably greater than or equal to 60% (60% or more) and better still 65% (65% or more).

Thus, one subject of the invention is a composition, preferably a cosmetic and/or dermatological composition, in the form of a water-in-oil emulsion comprising an aqueous phase dispersed in an oily phase and an amount, to stabilize the aqueous phase, of at least one amphiphilic 2-acrylamido-2-methylpropanesulphonic acid polymer or copolymer bearing hydrophobic grafts chosen from hydrocarbon-based radicals containing from 6 to 30 carbon atoms, oxyalkylenated hydrocarbon-based radicals comprising a hydrocarbon-based group containing from 6 to 30 carbon atoms and at least one oxyalkylene unit, perfluoroalkyl radicals, silicon radicals and mixtures thereof, the molar degree of grafting with hydrophoblic graft of the polymer or copolymer being at least equal to 50%.

A subject of the invention is also the use, in a composition such as a cosmetic and/or dermatological composition, in the form of a water-in-oil emulsion comprising an aqueous phase dispersed in an oily phase, as an agent for stabilizing the aqueous phase, of a stabilizing amount of at least one amphiphilic 2-acrylamido-2-methylpropanesulphonic acid polymer or copolymer bearing hydrophobic grafts chosen from hydrocarbon-based radicals containing from 6 to 30 carbon atoms, oxyalkylenated hydrocarbon-based radicals comprising a C₆-C₃₀ hydrocarbon-based group and at least one oxyalkylene unit perfluoroalkyl radicals, silicon radicals, and mixtures thereof, the molar degree of grafting with hydrophobic graft of the polymer or copolymer being at least equal to 50%.

The polymers and copolymers of the invention are liposoluble or lipo-dispersible derivatives of 2-acrylamido-2-methylpropanesulphonic acid (AMPS), bearing alkyl and/or alkylpolyoxyalkylenated grafts and/or perfluoroalkyl radical and/or silicon radical. The molar degree of grafting of these grafts is greater than or equal to 50%, preferably greater than or equal to 60% and better still greater than or equal to 65%, such as for example 70%, 75%, 80%, 85%, 90%, 95%, etc.

The expression “soluble or dispersible in a polar solvent polymers and copolymers” means polymers and copolymers which, when introduced into a polar solvent, at a concentration equal to 1% by weight, with stirring at 50° C. for 48 hours, give a macroscopically homogeneous solution and preferably whose light transmittance may be, at a wavelength equal to 500 nm through a sample 1 cm thick, is at least 50% and preferably at least 60%.

The expression “polar solvent” means a solvent having a dielectric constant measured at 25° C., less than 50. This solvent may be especially chosen from lower alcohols having from 1 to 6 carbon atoms, such as ethanol (dielectric constant: 24.6); ketones such as acetone (dielectric constant: 20.7); polyols such as propylene glycol (dielectric constant: 30.2); ethers such as diethylether (dielectric constant: 4.3), and their mixtures.

The polymers and copolymers in accordance with the invention are amphiphilic 2-acrylamido-2-methylpropanesulphonic acid (AMPS) polymers and copolymers.

The expression “amphiphilic polymer and copolymer” means any polymer or copolymer comprising both a hydrophilic portion and a hydrophobic portion.

The hydrophobic portion present in the polymers and copolymers of the invention corresponds to the lateral fatty chains or hydrophobic grafts.

The hydrophobic grafts are covalently linked to the main backbone of the polymer or copolymer, preferably via a functional bonding group, for example an ester, amide, ether or urethane group.

The hydrophobic grafts are chosen from C₆-C₃₀, preferably C₆-C₂₂ and better still C₁₂-C₁₈ hydrocarbon-based radicals, the hydrocarbon-based radicals defined above and also comprising at least one oxyalkylene unit, generally 1 to 30 oxyalkylene units, preferably 1 to 25 oxyalkylene units and better still from 3 to 20 oxyalkylene units, in particular oxyethylene or oxypropylene units, perfluoroalkyl radicals, silicon radicals, and mixtures of these units.

Included among the hydrocarbon-based radicals that are suitable for the hydrophobic grafts according to the invention are:

linear or branched alkyl radicals, for example n-hexyl, n-octyl, n-decyl, n-hexadecyl, n-dodecyl and isostearyl;

linear or branched alkenyl radicals, for example oleyl;

alicyclic radicals, for example cyclododecane and adamantane;

the cholesteryl radical and cholesterol ester residues, for example the cholesteryl oxyhexanoate group;

polycyclic aromatic radicals, for example naphthyl and pyrenyl radicals.

As perfluoroalkyl radicals, included in particular are C₆-C₁₈ radicals, for example the radical of formula:

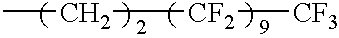

The silicon radicals include polydimethylsiloxanes (PDMS) having a molecular weight lower than 20,000 g/mole.

The hydrophobic grafts that are preferred are linear or branched, such as the n-dodecyl and n-hexadecyl radicals and isostearyl radicals, or alkenyl radicals such as oleyl.

Among the hydrocarbon-based radicals comprising 1 to 100 oxyalkylene units, mention may be made of the radicals of formula:

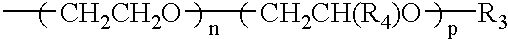

in which R₃ represents a C₆-C₃₀ hydrocarbon-based radical as defined above, R₄ represents hydrogen or a linear or branched C₁-C₆ alkyl group (preferably methyl) and n and p are, independently of each other, integers from 0 to 100, preferably 1 to 30 and better still from 3 to 20, with the proviso that the sum n+p, which is not zero, is less than or equal to 100, preferably less than 30 and better still less than 20.

The polymers and copolymers in accordance with the invention are preferably partially or totally neutralized with a mineral base (for example sodium hydroxide, potassium hydroxide or aqueous ammonia or ammonium substituted with from 1 to 4 alkyl groups having from 1 to 15 carbon atoms) or an organic base such as monoethanolamine, diethanolamine, triethanolamine, an aminomethylpropanediol, N-methylglucamine, basic amino acids, for instance arginine and lysine, and mixtures thereof.

The polymers and copolymers in accordance with the invention generally have a weight-average molecular weight ranging from 50,000 to 10,000,000 g/mole, more preferably from 100,000 to 8,000,000 g/mole and even more preferably from 100,000 to 7,000,000 g/mole.

The amphiphilic AMPS polymers and copolymers according to the invention may be crosslinked or non-crosslinked. They are preferably non-crosslinked.

The crosslinking agents include compounds containing olefinic polyunsaturation commonly used for crosslinking polymers obtained by free-radical polymerization. Examples that may be mentioned include divinylbenzene, diallyl ether, dipropylene glycol diallyl ether, polyglycol diallyl ethers, triethylene glycol divinyl ether, hydroquinone diallyl ether, tetraallyl-oxyethanoyl or other polyfunctional allyl or vinyl alcohol ethers, tetraethylene glycol diacrylate, triallylamine, trimethylolpropane diallyl ether, tetraallyloxyethane, methylenebisacrylamide, allylic ethers of alcohols of the sugar series, allyl methacrylate, trimethylolpropane triacrylate (TMPTA), or mixtures thereof.

Methylenebisacrylamide, allyl methacrylate or trimethylolpropane triacrylate (TMPTA) will be used more particularly. The degree of crosslinking can preferably range from 0.01 mol % to 10 mol % and more particularly from 0.2 mol % to 2 mol % relative to the polymer or copolymer.

The polymers in accordance with the invention may be chosen especially from random amphiphilic AMPS polymers modified by reaction with a C₆-C₂₂ n-monoalkylamine or di-n-alkylamine, such as those described in patent application WO-A-00/3 1154 (forming an integral part of the content of this description and being incorporated herein by reference). These polymers may also contain other ethylenically unsaturated hydrophilic monomers chosen, for example, from acrylic acid, methacrylic acid or alkyl derivatives thereof substituted in the β position or esters thereof obtained with mono- or polyalkylene glycols, acrylamide, methacrylamide, itaconic acid, styrene sulphonic acid, vinylsulphonic acid, (meth)allylsulphonic acid, vinylphosphonic acid, N-vinylacetamide, N-methyl N-vinylacetamide, N-vinylformamide, N-methyl N-vinylformamide, N-vinyllactames having an alkyle group having from 4 to 9 carbon atoms, such as N-vinylpyrrolidone, N-butyrolactame and N-vinylcaprolactame, maleic anhydride, acrylonitrile, glycidyl (meth)acrylate, vinyle chloride, vinylidene chloride, methyldiallylammonium chloride, quaternized dimethylaminoethyl methacrylate (MADAME), (meth)acrylamidopropyl-trimethylammonium chloride (APTAC and MAPTAC), methylvinylimidazolium chloride, or mixtures thereof.

The preferred copolymers of the invention are chosen from amphiphilic copolymers of AMPS and of at least one ethylenically unsaturated monomer comprising at least one hydrophobic portion containing from 6 to 30 carbon atoms, preferably from 6 to 22 carbon atoms, more preferably from 6 to 18 carbon atoms and more particularly from 12 to 18 carbon atoms.

These same copolymers can also contain one or more ethylenically unsaturated hydrophilic comonomers, for instance acrylic acid, methacrylic acid or alkyl derivatives thereof substituted in the P position or esters thereof obtained with mono- or polyalkylene glycols, acrylamide, methacrylamide, itaconic acid, styrene sulphonic acid, vinylsulphonic acid, (meth)allylsulphonic acid, vinylphosphonic acid, N-vinylacetamide, N-methyl N-vinylacetamide, N-vinylformamide, N-methyl N-vinylformamide, N-vinyllactames having an alkyle group having from 4 to 9 carbon atoms, such as N-vinylpyrrolidone, N-butyrolactame and N-vinylcaprolactame, maleic anhydride, acrylonitrile, glycidyle (meth)acrylate, vinyle chloride, vinylidene chloride, methyldiallylammonium chloride, quaternized dimethylaminoethyl methacrylate (MADAME), (meth)acrylamidopropyl-trimethylammonium chloride (APTAC et MAPTAC), methylvinylimidazolium chloride.

The ethylenically unsaturated hydrophobic monomers of these specific copolymers are preferably chosen from the acrylates and acrylamides of formula (1) below:

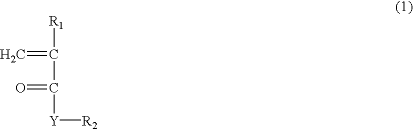

in which R₁ denotes a hydrogen atom, a linear or branched C₁-C₆ alkyl radical (preferably methyl); Y denotes O or NH or NH—CO—O; R₂ denotes a hydrophobic hydrocarbon-based radical containing from 6 to 22 carbon atoms, preferably from 6 to 18 and more particularly from 12 to 18 carbon atoms.

The hydrophobic radical R₂ is preferably chosen from C₆-C₁₈ alkyl and alkenyl radicals, that are linear (for example n-hexyl, n-octyl, n-decyl, n-hexadecyl, n-dodecyl or oleyl), branched (for example isostearyl) or alicyclic (for example cyclododecane or adamantane), C₆-C₁₈ perfluoroalkyl radicals, for example the group of formula

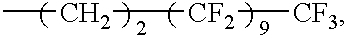

the cholesteryl radical or a cholesterol ester, for instance cholesteryl hexanoate; aromatic polycyclic groups, for instance naphthalene or pyrene. Among these radicals, the ones that are more particularly preferred are the linear and branched alkyl radicals.

According to one particularly preferred form of the invention, the hydrophobic radical R₂ also comprises at least one alkyleneoxide unit and preferably a polyoxyalkylenated chain. The polyoxyalkylenated chain preferably consists of ethyleneoxide units and/or of propyleneoxide units and even more particularly consists solely of ethyleneoxide units. The number of moles of oxyalkylenated units generally ranges from 1 to 100 mol and preferably from 1 to 30 mol and more preferably from 3 to 20 mol.

Among these copolymers that may be mentioned are copolymers comprising from 0 to 45 mol % of acrylamide units, from 5 to 50 mol % of partially or totally neutralized AMPS units and comprising at least 50 mol % of units having a (C₆-C₁₈)alkyl or polyoxyethylenated alkyl chain, preferably at least 60% and better still at least 65%.

Included are polymers comprising:

(a) from 5 to 50 mol % of 2-acrylamido-2-methylpropanesulphonic acid (AMPS) units of formula (2) below:

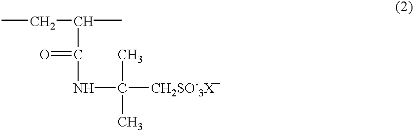

in which X⁺ is a proton, an alkali metal cation, an alkaline-earth metal cation, an ammonium ion or an ammonium ion substituted with from 1 to 4 alkyl groups having from 1 to 15 carbon atoms; and

(b) from 50 to 95 mol % of units of formula (3) below:

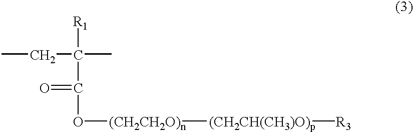

in which n and p, independently of each other, denote a number of moles ranging from 0 to 100, preferably from 1 to 30 and more preferably from 3 to 20, with the proviso that n+p is less than or equal to 100, preferably less than 30 and better still less than 20; R₁ has the same meaning indicated above for formula (1) and R₃ denotes a linear or branched alkyl containing from 6 to 22 carbon atoms, preferably from 6 to 18 carbon atoms and better still from 12 to 18 carbon atoms.

The stabilizing amount of polymer or copolymer is any amount that is sufficient to give the W/O emulsion the required stability, in particular that is sufficient to avoid the phenomenon of flocculation of the emulsion (destabilization).

The amount of the polymers and copolymers used in accordance with the invention is not limited. These polymers and copolymers are generally present in the invention compositions in concentrations by weight ranging from 0.1% to 10%, preferably from 0.5 to 8% and better still from 1% to 5% relative to the total weight of the composition.

The non-flocculated nature of the emulsions may be demonstrated by optical microscopy, after dilution, if necessary, when the emulsions are concentrated. A non-flocculated emulsion consists of drops that are independent of each other; a flocculated emulsion comprises aggregates of drops, which is not observed in the compositions of the invention.

The fatty phase or oily phase usually contains at least one oil. As oils that may be used in the composition of the invention, mention may be made, for example, of: hydrocarbon-based oils of animal origin, such as perhydrosqualene;

hydrocarbon-based oils of plant origin, such as liquid triglycerides of fatty acids containing from 4 to 22 carbon atoms, for instance heptanoic or octanoic acid triglyceride or alternatively, for example, sunflower oil, corn oil, soybean oil, marrow oil, grapeseed oil, sesame oil, hazelnut oil, apricot oil, macadamia oil, arara oil, sunflower oil, castor oil, avocado oil, caprylic/capric acid triglycerides for instance those sold by the company Stearineries Dubois or those sold under the names Miglyol 810, 812 and 818 by the company Dynamit Nobel, jojoba oil or karite butter oil;

synthetic esters and synthetic ethers, especially of fatty acids, for instance oils of formulae R′COOR″ and R′OR″ in which R′ represents a fatty acid residue containing from 8 to 29 carbon atoms and R″ represents a branched or unbranched hydrocarbon-based chain containing from 3 to 30 carbon atoms, such as, for example, purcellin oil, isononyl isononanoate, isopropyl myristate, 2-ethylhexyl palmitate, 2-octyldodecyl stearate, 2-octyldodecyl erucate or isostearyl isostearate; hydroxylated esters such as isostearyl lactate, octyl hydroxystearate, octyldodecyl hydroxystearate, diisostearyl malate, triisocetyl citrate and fatty alcohol heptanoates, octanoates and decanoates; polyol esters, for instance propylene glycol dioctanoate, neopentyl glycol diheptanoate and diethylene glycol diisononanoate; and pentaerythritol esters, for instance pentaerythrityl tetraisostearate;

linear or branched hydrocarbons of mineral or synthetic origin, such as volatile or non-volatile liquid paraffins, and derivatives thereof, petroleum jelly, polydecenes, and hydrogenated polyisobutene such as Parleame oil;

fatty alcohols containing from 8 to 26 carbon atoms, for instance cetyl alcohol, stearyl alcohol and a mixture thereof (cetylstearyl alcohol), octyldodecanol, 2-butyloctanol, 2-hexyldecanol, 2-undecylpentadecanol, oleyl alcohol or linoleyl alcohol;

alkoylated and especially ethoxylated fatty alcohols or fatty acids, such as oleth-12 and PEG-20 stearate;

partially hydrocarbon-based and/or silicone-based fluoro oils, for instance those described in document JP-A-2 295 912. Examples of fluoro oils which may also be mentioned include perfluoromethylcyclopentane and perfluoro-1,3-dimethylcyclohexane, sold under the names “Flutec PC1®” and “Flutec PC3®” by the company BNFL Fluorochemicals; perfluoro-1,2-dimethylcyclobutane; perfluoroalkanes such as dodecafluoropentane and tetradecafluorohexane, sold under the names “PF 5050®” and “PF 5060®” by the company 3M, or alternatively bromoperfluorooctyl sold under the name “Foralkyl®” by the company Atochem; nonafluoromethoxybutane sold under the name “MSX 4518®” by the company 3M and nonafluoroethoxyisobutane; perfluoromorpholine derivatives, such as the 4-trifluoromethylperfluoromorpholine sold under the name “PF 5052®” by the company 3M;

silicone oils, for instance volatile or non-volatile polymethylsiloxanes (PDMSs) containing a linear or cyclic silicone chain, that are liquid or pasty at room temperature, especially cyclopolydimethylsiloxanes (cyclomethicones) such as cyclohexasiloxane; polydimethylsiloxanes comprising alkyl, alkoxy or phenyl groups, that are pendent or at the end of a silicone chain, these groups containing from 2 to 24 carbon atoms; phenylsilicones, for instance phenyltrimethicones, phenyldimethicones, phenyltrimethylsiloxy-diphenylsiloxanes, diphenyldimethicones, diphenylmethyldiphenyltrisiloxanes, 2-phenylethyltrimethylsiloxysilicates and mixtures thereof.

In the list of oils mentioned above, the expression “hydrocarbon-based oil” means any oil mainly comprising carbon and hydrogen atoms, and optionally ester, ether, fluoro, carboxylic acid and/or alcohol groups.

Other fatty substances that may be present in the oily phase include, for example, fatty acids containing from 8 to 30 carbon atoms, for instance stearic acid, lauric acid, palmitic acid and oleic acid; waxes, for instance lanolin, beeswax, carnauba wax or candelilla wax, paraffin wax, lignite wax or microcrystalline waxes, ceresin or ozokerite, synthetic waxes such as polyethylene waxes, Fischer-Tropsch waxes; gums such as silicone gums (dimethiconol); silicone resins such as trifluoromethyl-C₁₋₄-alkyldimethicone and trifluoropropyldimethicone; and silicone elastomers, for instance the products sold under the names “KSG” by the company Shin-Etsu, under the names “Trefil”, “BY29” or “EPSX” by the company Dow Corning or under the names “Gransil” by the company Grant Industries and other known and yet to be discovered gelifying agents of an oily phase.

These fatty substances may be chosen in a varied manner by a person skilled in the art so as to prepare a composition having the desired properties, for example in terms of consistency or texture.

The amount of oily phase in the emulsion of the invention is not limited and can range, for example, from 2% to 5%, preferably from 5% to 50% and better still from 10% to 40% by weight relative to the total weight of the composition.

In a known manner, the composition of the invention can also contain adjuvants that are new or common in cosmetics and/or dermatology, other than those mentioned above, such as solvents, active agents, preserving agents, antioxidants, complexing agents, fragrances, organic or mineral UV filters (screening agents), salts, fillers, bactericides, odour absorbers, dyestuffs and lipid vesicles. The amounts of these various adjuvants may be those conventionally used in the field under consideration, and, for example, from 0.01% to 20% relative to the total weight of the composition. Depending on their nature, these adjuvants may be introduced into the fatty phase, into the aqueous phase and/or into the lipid vesicles. Needless to say, a person skilled in the art will take care to select the optional additional additives and/or the amount thereof such that the advantageous properties of the composition according to the invention are not, or are not substantially, adversely affected by the envisaged addition.

Active agents that may be used in the composition of the invention include, for example, moisturizers and, for example, protein hydrolysates and polyols such as glycerol, glycols, for instance polyethylene glycols, and sugar derivatives; natural extracts; procyannidol oligomers; vitamins, for instance vitamin A (retinol), vitamin C (ascorbic acid), vitamin E (tocopherol), vitamin B5 (panthenol) and vitamin B3 (niacinamide); urea; caffeine; depigmenting agents such as cogic acid and caffeic acid; salicylic acid; α-hydroxy acids such as lactic acid and glycolic acid; retinoids such as carotenoids; hydrocortisone; melatonin; extracts of algae, of fungi, of plants, of yeasts or of bacteria; hydrolysed, partially hydrolysed or unhydrolysed proteins, and enzymes; DHEA and derivatives and metabolites thereof; antibacterial active agents for treating greasy skin, for instance 2,4,4′-trichloro-2′-hydroxydiphenyl ether (or tricolosan) and 3,4,4′-trichlorocarbanilide (or triclocarban); matt-effect agents, for instance fibres; sunscreens, and mixtures thereof.

As organic UV sunscreens, included are, for example:

Butylmethoxydibenzoylmethane sold especially by HOFFMANN LA ROCHE under the name PARSOL 1789,

octocrylene sold especially by BASF under the name UVINUL N539,

octylsalicylate sold especially by Haarman-Reimer under the name Neo Heliopan OS,

octylmethoxycinnamate sold especially by Hoffmann Laroche under the name PARSOL MCX,

phenylbenzimidazole sulphonic acid sold especially by Merck under the name Eusolex 232,

oxybenzones such as benzophenones-3, -4 ou -5,

benzotriazoles silicones and in particular drometrizole trisiloxane, and those described in the document FR-A-2,642,968,

terephtalylidene di-camphor sulphonic, in particular Mexoryl SX sold by Chimex,

and the insoluble organic sunscreens such as triazine derivatives, and, in particular 2,4-bis ([4-(2-ethyl-hexyloxy)-2-hydroxy]-phenyl)-6-(4-methoxy-phenyl)-1,3,5-triazine sold by CIBA GEIGY under the name TINOSORB S, and 2,2′-methylenebis-[6-(2H benzotriazol-2-yl) 4-(1,1,3,3-tetramethylbutyl)-phenol] sold by CIBA GEIGY under the name TINOSORB M.

As physic UV sunscreens, mention may be made, for example, of titan or zinc oxides, as microparticules or nanoparticules (nanopigments) which are eventually coated.

The active agent(s) may be present, for example, in a concentration ranging from 0.01% to 20%, preferably from 0.1% to 10% and better still from 0.5% to 5% relative to the total weight of the composition.

Useful fillers which may be used in the composition of the invention include for example, besides pigments, silica powder; talc; polyamide particles and especially those sold under the name Orgasol by the company Atochem; polyethylene powders; microspheres based on acrylic copolymers, such as those based on ethylene glycol dimethacrylate/lauryl methacrylate copolymer sold by the company Dow Corning under the name Polytrap; expanded powders such as hollow microspheres and especially the microspheres sold under the name Expancel by the company Kemanord Plast or under the name Micropearl F 80 ED by the company Matsumoto; powders of natural organic materials such as crosslinked or noncrosslinked corn starch, wheat starch or rice starch, such as the powders of starch crosslinked with octenyl succinate anhydride, sold under the name Dry-Flo by the company National Starch; silicone resin microbeads such as those sold under the name Tospearl by the company Toshiba Silicone; latex; and mixtures thereof. These fillers may be present in amounts ranging from 0.01% to 20% by weight and preferably from 0.1% to 10% by weight and better 0.5 A 5% by weight relative to the total weight of the composition.

The composition of the invention may eventually contain also surfactants, for example cleansing surfactants (which are foaming surfactants and not emulsifying surfactants) and especially non ionic surfactants such as ethoxylated or glycerolated fatty alcohols or acids; anionic surfactants such as alkyl ether sulphonates or alkyl ether sulphates, or phosphate esters or ethers; cationic surfactants such as salts of cationic fatty acid such as Esterquats; amphoteric and zwitterionic surfactants such as betaines; and mixtures thereof.

Furthermore, the composition of the invention may contain in the aqueous phase, one or several hydrophilic polymers chosen for example from carboxyvinylic polymers such as carbopols (carbomers); polymers of 2-acrylamido-2-methylpropane sulphonic acid which are soluble or dispersible in aqueous phase such as the polymer marketed under the name <<Hostacerin AMPS>> (INCI name: ammonium polyacryldimethyl-tauramide), by Clariant; synthetic neutral polymers such as polyvinylpyrrolidone (PVP), polyvinyl acetate (PVA); polysaccharides such as guar or xanthane gums and cellulosic derivatives; hydrosoluble ou hydrodispersible silicone derivatives such as acrylic silicones and cationic silicones; and mixtures thereof. The polymer or the polymers may be present in a concentration from 0.01 to 20%, preferably from 0.05 to 10% and better from 0.1 to 5% of the total weight of the composition.

The compositions according to the invention are preferably in the form of water-in-oil emulsions of liquid to semi-liquid consistency, such as milks, more or less unctuous creams, cream-gels or pastes. They may optionally be packaged as aerosols and may be in the form of a mousse or a spray.

Advantageously, according to one particular embodiment of the invention, the emulsions prepared with the polymers and/or copolymers according to the invention may comprise only 1% by weight or less, or may even be free of, emulsifying surfactants, while at the same time being stable on storage.

The composition according to the invention may be used in many cosmetic or dermatological applications, and in particular it may be used to treat, care for and/or makeup and/or cleanse facial and/or body skin, mucous membranes (lips), the scalp and/or keratin fibres (hair or eyelashes), for example to give the face a light and radiant complexion, a healthy look, a smooth and younger appearance, to treat skin wrinkles and fine lines, to depigment the skin and especially to remove age marks, to eliminate comedones, to treat damaged hair, tonify it and restore its vitality, and to strengthen keratin fibres.

Thus, the compositions of the invention may be used as care and/or hygiene products, such as protective, treatment or care creams for the face, for the hands or for the body, protective or care body milks, lotions, gels or mousses to care for the skin and mucous membranes or to cleanse (including makeup removal) or scrub the skin. They may also constitute make-up products for keratin fibres, for the skin, for the lips and/or for the nails. They may especially constitute a foundation, a face powder, an eyeshadow, a lipstick, a mascara or an eyeliner.

The compositions of the invention may also be used as antisun products for protecting the skin against UV rays.

The compositions according to the invention may be used as rinse-out or leave-in hair products, especially for washing, caring for, conditioning or maintaining the hairstyle or for shaping keratin fibres such as the hair. They may also consist of solid preparations constituting soaps or cleansing bars.

The compositions of the invention may also be used as buccodental care products, such as toothpastes.

They may also be styling products such as hairsetting lotions, blow-drying lotions and fixing or styling compositions. The lotions may be packaged in various forms, especially in vaporizers, pump-dispenser bottles or in aerosol containers so as to be able to apply the composition in vaporized form or in the form of a mousse. Such packaging forms are indicated, for example, when it is desired to obtain a spray or a mousse for fixing or treating the hair.

Thus, a subject of the present invention is also the cosmetic use of the composition according to the invention to treat, care for, make up and/or cleanse facial and/or body skin, mucous membranes (lips), the scalp and/or keratin fibres.

Another subject of the invention is a cosmetic process for treating human keratin materials such as the skin, including the scalp, the hair, the eyelashes, the eyebrows, the nails or mucous membranes, especially the lips, characterized in that a cosmetic composition as defined above is applied to the keratin materials, according to the usual technique for using this composition. For example, application of creams, gels, sera, lotions or milks to the skin, the scalp and/or mucous membranes. The type of treatment depends on the active agent(s) present in the composition.

A subject of the invention is also a use of the above composition to prepare a salve or an ointment for treating the human face and/or body, including the hands, especially to treat acne.

Finally, a method of preparing the invention compositions is also a subject of the invention, and comprises mixing together the invention (co)polymer(s) and the emulsion components in any order of addition or contact.

The examples that follow illustrate the present invention but do not limit it. In the examples, except where otherwise indicated, all the percentages and parts are expressed on a weight basis.

### EXAMPLE 1

### Care Cream

In this example, an AMPS copolymer comprising (C₁₂₋₁₄) alkyl grafts containing 3 oxyethylenated units (molar degree of grafting=69%) is used, a 1% solution of which in a mixture of tocopheryl acetate (13.3% by weight) and 60/40 C₈₋₁₀ caprylic/capric triglycerides (86.7%) has a light transmittance, at a wavelength of 500 nm, equal to 68%. This transmittance measurement is carried out using a Cary 300 spectrophotometer (Varian), using cuvettes 1 cm thick.

| | % |
|---|---|
| Oily phase | |
| AMPS copolymer comprising C₁₂₋₁₄ grafts, 3 oxyethylenated units | 2.5 |
| (molar degree of grafting = 69%) | |
| Tocopheryl acetate | 3 |
| 60/40 C₈₋₁₀ caprylic/capric triglycerides | 19.5 |
| Aqueous phase | |
| Glycerol | 3 |
| Magnesium sulphate | 2.25 |
| Preserving agent | 0.07 |
| Demineralized water | 69.68 |

Preparation Method

The AMPS amphiphilic copolymer is dissolved for 48 hours with stirring in the oily phase at 50° C.; the solution obtained is macroscopically homogeneous. The emulsion is prepared by slowly introducing the aqueous phase into the oily phase with stirring using a Moritz homogenizer at a stirring speed of 1500 rpm.

The size of the drops in the emulsion obtained is between 100 nm and 2 microns. The viscosity at a shear rate of 200 s⁻¹ is equal to 1.4 Pa.s (measured using a Haake RS 150 rheometer equipped with a 6 cm/2° cone/plate geometry at about 25° C.).

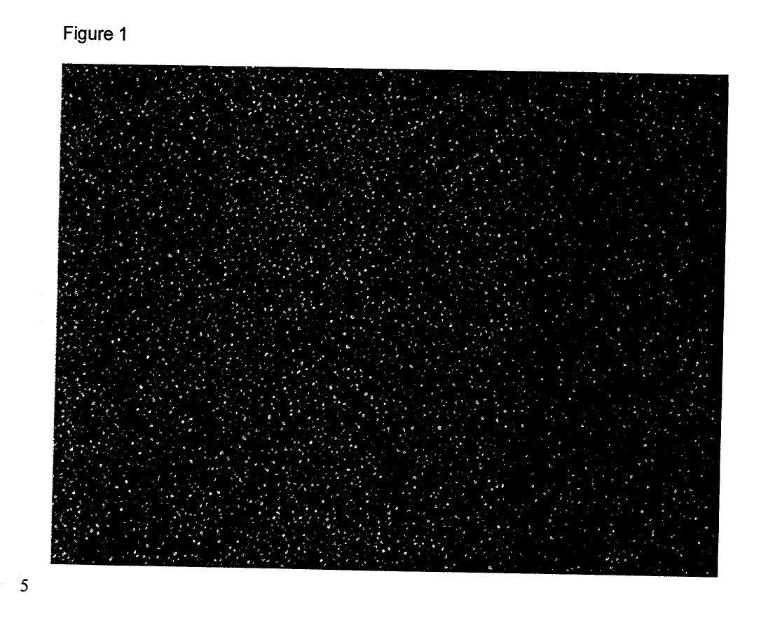

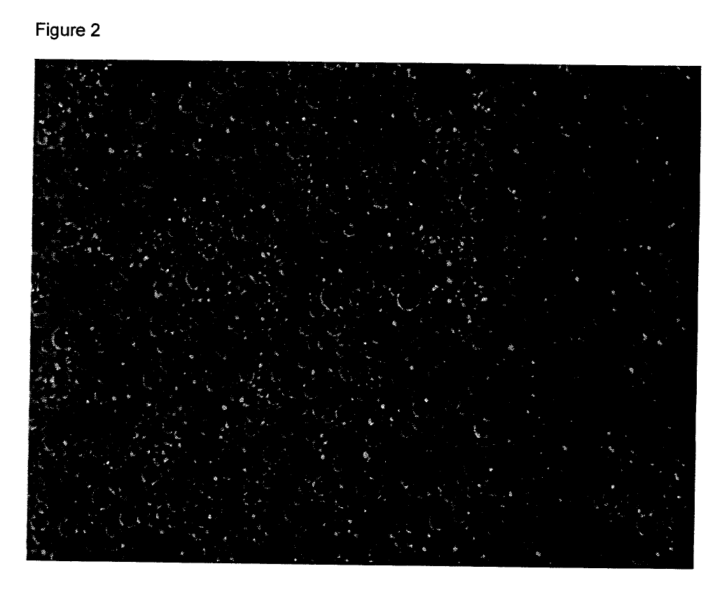

FIGS. 1 and 2 are photographs of the water-in-oil emulsion obtained, taken using an optical microscope in interference contrast mode, at magnifications of ×400 (FIG. 1) and ×1 000 (FIG. 2).

As shown in FIGS. 1 and 2, the emulsion is not flocculated, and has a beautiful texture of cream type, that is relatively non-greasy and pleasant to apply.

### EXAMPLE 2

### Milk for Dry Skin

In this example, an AMPS copolymer comprising C₁₂₋₁₄ grafts and 3 oxyethylenated units (molar degree of grafting=69%) is used, a 1% solution of which in a mixture of tocopheryl acetate (13.3% by weight) and 60/40 C₈₋₁₀ caprylic/capric triglycerides (86.7%) has a light transmittance, at a wavelength of 500 nm, equal to 68%. This transmittance measurement is carried out using a Cary 30 spectrophotomer (Varian), using cuvettes 1 cm thick.

| | % |
|---|---|
| Oily phase | |
| AMPS copolymer comprising C₁₂₋₁₄ grafts, 3 oxyethylenated units | 2.5 |
| (molar degree of grafting = 69%) | |
| Tocopheryl acetate | 3 |
| 60/40 C₈₋₁₀ caprylic/capric triglycerides | 25.5 |
| Oily gelling agent | 4 |
| Aqueous phase | |
| Glycerol | 3 |
| Magnesium sulphate | 1.95 |
| Preserving agent | 0.06 |
| Demineralized water | 59.99 |

Preparation Method

The AMPS amphiphilic copolymer is dissolved for 48 hours with stirring in the oily phase at 50° C.; the solution obtained is macroscopically homogeneous. The emulsion is prepared by slowly introducing the aqueous phase into the oily phase with stirring using a Moritz homogenizer at a stirring speed of 1500 rpm.

The size of the drops is about 1 micron. The viscosity at a shear rate of 200 s⁻¹ is equal to 0.6 Pa.s (measured using a Haake RS 150 rheometer equipped with a 6 cm/2° cone/plate geometry at about 25° C.).

The emulsion is not flocculated and has a beautiful fluid texture suitable for a body milk, of dry-skin type.

### COMPARATIVE EXAMPLE

In this counter-example, an AMPS copolymer comprising isostearic grafts and 20 oxyethylenated units (molar degree of grafting=40.8%) is used, a 1% solution of which in a mixture of tocopheryl acetate (13.3% by weight) and 60/40 C₈₋₁₀ caprylic/capric triglycerides (86.7%) has a light transmittance, at a wavelength of 500 nm, equal to 2%. This transmittance measurement is carried out using a Cary 300 spectrophotomer (Varian), using cuvettes 1 cm thick.

| | % |
|---|---|
| Oily phase | |
| AMPS copolymer comprising isostearic grafts and | 2.5 |
| 20 oxyethylenated units (molar degree of grafting = 40.8%) | |
| Tocopheryl acetate | 3 |
| 60/40 C₈₋₁₀ caprylic/capric triglycerides | 24.5 |
| Oily gelling agent | 4 |
| Aqueous phase | |
| Glycerol | 3 |
| Preserving agent | 0.07 |
| Demineralized water | 66.93 |

Preparation Method

The AMPS amphiphilic copolymer is introduced for 48 hours with stirring into the oily phase at 50° C.; the solution obtained contains undissolved polymer aggregates, that can be seen visually. After 24 hours at rest, this solution contains sedimented polymer aggregates. The emulsion is prepared by slowly introducing the aqueous phase into the oily phase with stirring using a Moritz homogenizer at a stirring speed of 1500 rpm.

The emulsion obtained is of oil-in-water type, rather than water-in-oil type as in the examples according to the invention.

French Patent Application 0107609 filed Jun. 11, 2001 is incorporated herein by reference, as are all documents, texts, standards, references, patents and applications referred to above.

1. A composition in the form of a water-in-oil emulsion comprising an aqueous phase dispersed in an oily phase and an aqueous phase stabilizing amount of at least one amphiphilic 2-acrylamido-2-methylpropanesulphonic acid polymer or copolymer bearing hydrophobic grafts selected from hydrocarbon-based radicals containing from 6 to 30 carbon atoms, oxyalkylenated hydrocarbon-based radicals comprising a hydrocarbon-based group containing from 6 to 30 carbon atoms and at least one oxyalkylene unit, perfluoroalkyl radicals, silicon radicals, and mixtures thereof, the molar degree of grafting with hydrophobic grafts of the polymer or copolymer being at least equal to 50%.

2. The composition according to claim 1, wherein the molar degree of grafting of the polymer or copolymer is greater than or equal to 60%.

3. The composition according to claim 1, wherein the hydrocarbon-based radicals of the hydrophobic grafts contain from 6 to 22 carbon atoms.

4. The composition according to claim 1, wherein the hydrocarbon-based radicals are selected from the group consisting of linear or branched alkyl radicals, linear or branched alkenyl radicals, alicyclic radicals, the cholesteryl radical and cholesterol ester residues, and polycyclic aromatic radicals.

5. The composition according to claim 1, wherein the hydrophobic grafts are oxyalkylenated hydrocarbon-based radicals comprising a hydrocarbon-based group containing 6 to 22 carbon atoms and 1 to 100 oxyalkylene units.

6. The composition according to claim 5, wherein the oxyalkylenated hydrocarbon-based radicals correspond to the formula: 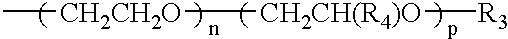
in which R₃ represents a C₆-C₃₀ hydrocarbon-based radical, R₄ represents hydrogen or a linear or branched C₁-C₆ alkyl group and n and p are, independently of each other, integers from 0 to 100, with the proviso that the sum n+p, which is not zero, is less than or equal to 100.

7. The composition according to claim 1, wherein the hydrophobic grafts are covalently linked to the main chain of the polymer or of the copolymer.

8. The composition of claim 7, wherein said hydrophobic grafts are covalently linked via a functional bonding group chosen from ester, amide, ether and urethane groups.

9. The composition according to claim 1, wherein the amphiphilic copolymer containing hydrophobic grafts is a copolymer of 2-acrylamido-2-methylpropanesulphonic acid and of at least one ethylenically unsaturated monomer comprising a hydrophobic portion chosen from hydrocarbon-based radicals containing from 6 to 30 carbon atoms, and oxyalkylenated hydrocarbon-based radicals comprising a hydrocarbon-based radical as defined above, perfluoroalkyl radicals and silicon radicals, and at least one oxyalkylene unit.

10. The composition according to claim 9, characterized in that the ethylenically unsaturated hydrophobic monomer corresponds to the formula: 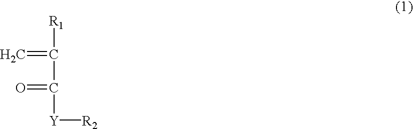
in which R₁ denotes a hydrogen atom, a linear or branched C₁-C₆ alkyl radical; Y denotes O or NH or NH—CO—O; R₂ denotes a hydrophobic hydrocarbon-based radical containing from 6 to 22 carbon atoms optionally further comprising 1 to 100 mol of oxyalkylenated units.

11. The composition according to claim 9, wherein the amphiphilic copolymer is also a copolymer of at least one other ethylenically unsaturated hydrophilic comonomer.

12. The composition according to claim 11, characterized in that the hydrophilic comonomer is selected from the group consisting of acrylic acid, methacrylic acid or alkyl derivatives thereof substituted in the β position or esters thereof obtained with mono- or polyalkylene glycols, acrylamide, methacrylamide, itaconic acid, styrene sulphonic acid, vinylsulphonic acid, (meth)allylsulphonic acid, vinylphosphonic acid, N-vinylacetamide, N-methyl N-vinylacetamide,.N-vinylformamide, N-methyl N-vinylformamide, N-vinyllactames having an alkyle group having from 4 to 9 carbon atoms, maleic anhydride, acrylonitrile, glycidyl (meth)acrylate, vinyle chloride, vinylidene chloride, methyldiallylammonium chloride, quaternized dimethylaminoethyl methacrylate, (meth)acrylamidopropyl-trimethylammoniumchloride, and methylvinylimidazolium chloride.

13. The composition according to claim 1, wherein the amphiphilic copolymer containing hydrophobic grafts comprises:
0 to 45 mol % of acrylamide units;
5 to 50 mol % of partially or totally neutralized AMPS units; and
50 mol % or more of n(C₆-C₁₈)alkyl or polyoxyethylenated alkyl units.

14. The composition according to claim 1, wherein the amphiphilic copolymer containing hydrophobic grafts consists of:
(a) from 5 to 50 mol % of 2-acrylamido-2-methylpropanesulphonic acid (AMPS) units of formula (2) below: 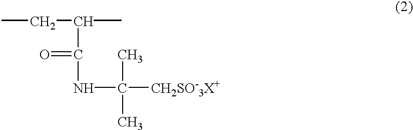
in which X⁺ is a proton, an alkali metal cation, an alkaline-earth metal cation, an ammonium ion or an ammonium ion substituted with from 1 to 4 alkyl groups having from 1 to 15 carbon atoms; and
(b) from 50 to 95 mol % of units of formula (3) below: 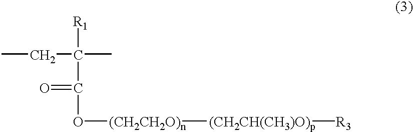
in which n and p, independently of each other, denote a number of moles ranging from 0 to 100 with the proviso that n+p is less than or equal to 100; R₁ is a hydrogen atom or a linear or branched C₁-C₆ alkyl radical and R₃ denotes a linear or branched alkyl containing from 6 to 22 carbon atoms.

15. The composition according to claim 1, wherein the amount of polymer or copolymer represents 0.1% to 10% by weight relative to the total weight of the composition.

16. The composition according to claim 1, characterized in that the amphiphilic polymer or copolymer is in free or partially or totally neutralized form.

17. The composition according to claim 1, wherein the amphiphilic polymer or copolymer has a weight-average molecular weight of from 50 000 to 10 000 000 g/mole.

18. The composition according to claim 1, wherein the amphiphilic polymer or copolymer is not crosslinked.

19. The composition according to claim 1, further comprising 1% by weight or less of emulsifying surfactants relative to the total weight of the composition.

20. The composition according to claim 1, comprising no emulsifying surfactants.

21. The composition according to claim 1, wherein the oily phase represents 2% to 60% by weight relative to the total weight of the composition.

22. The composition according to claim 1, wherein the oily phase comprises at least 5% by weight of hydrocarbon-based oils relative to the total weight of the composition.

23. A method to treat, care for, make up, and/or cleanse facial and/or body skin, mucous membranes, the scalp and/or keratin fibers, comprising applying the composition of claim 1 to the skin, mucous membranes, scalp and/or keratin fibers.

24. The method according to claim 23, wherein the composition comprises no emulsifying agents.

25. The method according to claim 23, wherein the amount of polymer or copolymer represents 0.1% to 10% by weight relative to the total weight of the composition.

26. The method according to claim 23, wherein said composition comprises 1% by weight or less of emulsifying surfactants relative to the total weight of the composition.

27. The method according to claim 23, wherein said composition comprises no emulsifying surfactants.

28. The method according to claim 23, wherein the oily phase represents 2% to 60% by weight relative to the total weight of the composition.

29. The method according to claim 23, wherein the oily phase comprises at least 5% by weight of hydrocarbon-based oils relative to the total weight of the composition.

30. The method of claim 23, wherein said composition is applied to the keratin fibers.

31. The method of claim 23, wherein said composition is applied to the skin.
